(19) United States
(12) Patent Application Publication
(10) Pub. No.: US 2025/0064724 A1
(43) Pub. Date: Feb. 27, 2025

(54) MODULAR FIELD-STABLE BIO-ARTIFICIAL BLOOD SUBSTITUTE

(71) Applicant: VIRGINIA COMMONWEALTH UNIVERSITY, Richmond, VA (US)

(72) Inventors: Martin J. MANGINO, Powhatan, VA (US); Loren K. LIEBRECHT, Richmond, VA (US); Jad KHORAKI, Richmond, VA (US)

(21) Appl. No.: 18/718,963

(22) PCT Filed: Nov. 18, 2022
(86) PCT No.: PCT/US2022/050366

§ 371 Date: Jun. 12, 2024

Related U.S. Application Data
(60) Provisional application No. 63/280,808, filed on Nov. 18, 2021.

(51) Int. Cl. A61K 9/00; A61K 31/198; A61K 38/36; A61K 47/02; A61K 47/10; A61P 7/00

(52) U.S. Cl. CPC A61K 9/0026; A61K 31/198; A61K 38/363; A61K 47/02; A61K 47/10; A61P 7/00

(57) ABSTRACT

Bio-artificial blood substitutes are provided for intravenous medical use to restore or increase tissue perfusion and oxygen delivery in a pre-hospital or hospital setting. The bio-artificial blood substitutes comprise three modular components (polyethylene glycol (PEG) polymers to increase perfusion, a hemoglobin-based oxygen carrier (HBOC) plus L-arginine to prove oxygen carrying capacity, and a lyophilized fibrinogen or plasma component to provide hemostasis potential, all of which are administered sequentially in modules to treat low perfusion states with possible coagulopathy as seen in blood loss and shock. The three solutions are stable at ambient temperature and are well-suited for use in the field, e.g., when immediate, critical care is needed such as on the battlefield, during natural disasters or other mass casualty events. Other use may include clinical need for transfusion or for peri-surgical resuscitation in patients unable to receive standard blood products.

### STATEMENT OF FEDERALLY SPONSORED RESEARCH AND DEVELOPMENT

This invention was made with government support under grant number BA150349 awarded by the Department of Defense. The United States government has certain rights in the invention.

### BACKGROUND OF THE INVENTION

### Technical Field

The invention generally relates to improved bio-artificial blood substitutes for intravenous medical use to restore or increase tissue perfusion and oxygen delivery. In particular, the invention provides stable, bio-artificial blood substitutes and methods of their use by sequential administration of modular components of the blood substitutes, including: synthetic high and low molecular weight polyethylene glycol (PEG) polymers, a hemoglobin-based oxygen carrier (HBOC) plus L-arginine, and, optionally, hemostasis components like fibrinogen or lyophilized plasma.

### Description of Related Art

The direct and indirect effects of severe and prolonged tissue hypoxia due to hemorrhagic shock are leading causes of death following battlefield injuries (1, 2). Resuscitation in the field is often seriously inadequate even in patients transported to local field hospitals for full resuscitation. Delays in evacuation in far-forward units may take many hours to days. Time becomes critical. Maintaining patients in a low-volume state for long periods is a real possibility (3). In 2016, civilian deaths due to injury in the US numbered over 190,000 with greater than $400 billion in yearly health care costs along with lost productivity (4). For US citizens under 44 years, trauma is the number one cause of death. For all age groups in the US, trauma is the third leading cause of death. Trauma accounts for approximately 20% of all life-years lost, compared to cancer (17%), heart disease (13%), and suicide/homicide (11%) (5). For traumatic injuries, hemorrhagic shock causes over 35% of pre-hospital deaths and over 40% of deaths within the first 24 hours. This is second only to trauma deaths induced by severe CNS injury (6). Hemorrhagic shock exposes patients to immediate complications of life-threatening infections, coagulopathies, and multiple organ failure (7, 8). Crystalloid-based I.V. solutions like saline or lactated Ringer's are available for pre-hospital use as non-blood volume expanders. They can be safely transported and stored but are generally limited in their effectiveness of increasing tissue perfusion or oxygenation as crystalloids increase blood volume and therefore flow for a limited time, but have no oxygen carrying capacity, do not maintain oncotic pressure, and diffuse out of the vascular space with an intravascular half-life of 20-30 minutes. Attempts to modify basic intravenous crystalloids for pre-hospital resuscitation by adding hypertonic NaCl or starch (Hextend®) as a volume expander have produced disappointing results (9, 10). There remains a need for a more effective resuscitation crystalloid with oxygen carrying capacity as well as increased tissue perfusion for patients with severe hemorrhagic shock, especially in pre-hospital settings where standard blood products are not available.

Additionally, there are non-traumatic situations where the need of a modular blood substitute would be advantageous and life-saving, such as in patients who require frequent transfusions (e.g. sickle cell anemia, hemophilia, hematologic cancers), elect not to receive traditional blood transfusions (e.g. Jehovah's witnesses), or have a surgery or procedure that may require a high volume of blood replacement. The top twenty surgical procedures that are associated with the highest risk for blood transfusion (and account for more than 50% of surgical patients exposed to transfusion) represent routine operations across many surgery disciplines, including: cardiovascular, hepatobiliary, colorectal, general surgery, surgical oncology, urological cancer, orthopedic, spinal, and gynecological (11). There will always be a need to limit patient blood loss and find safe, cost-effective, and flexible transfusion options for patients with various needs in multiple settings. This is mainly due to adverse effects of blood transfusion reactions, costs, and potential unavailability of product, as seen recently with COVID (12).

Attempts to freeze dry blood products including red blood cells, platelets, and plasma in order to provide oxygen carrying capacity in shock or blood loss have been attempted for decades with mixed results. The obvious benefits of storage stability have compelled much work in this area. In 2022, there are 3 major freeze-dried plasma products produced by France, Germany, and South Africa that have undergone trials in field trauma (13). These specific solutions and others provide blood clotting factors including fibrinogen by reconstituting the dried residue with sterile water in the field or hospital setting. These products generally perform the same as fresh frozen plasma in patient outcomes and in restoring coagulation in many trauma trials with similar side effect profiles (13, 14). Alternatively, perfluorocarbon-based oxygen-carrying products that have the capacity to dissolve oxygen in their chemical formulation with benefits of shelf storage stability have also been studied and developed for years with mixed results to aid in formation of a stable blood substitute.

### SUMMARY OF THE INVENTION

The deployable product described herein comprises two components or “modules” having different compositions, which may be given with or without a third module. The components must be administered in a defined order and are highly innovative in several ways.
1) A first-in class, novel, stable, effective, low volume, crystalloid impermeant and anti-shock therapeutic solution is provided that logarithmically improves field resuscitation of patients with severe tissue ischemia from hypovolemic shock due to trauma (15). The new solution uses a mixture of polyethylene glycols (PEGs) to target metabolic cell and tissue swelling, a root mechanism of tissue reperfusion injury. The current disclosure uses the active impermeant molecule with an intermediate osmotic reflection coefficient (0.2-0.8) that dramatically amplifies the impermeant osmotic effect in shock (16-19). These are polymers of polyethylene glycol with average molecular weights between 18,000-100,000. A smaller molecular weight PEG ranging from 1,000 to 18,000 is used to decrease capillary rouleaux formation and aid in impermeant based movement of water out of swollen, ischemic cells. The result is a powerful solution used in low volume resuscitation that restores capillary perfusion, increases oxygen transfer, and accelerates oxygen debt repayment in shock. This improves outcomes, including shock tolerance, increased safe pre-hospital transport times, and survival, multiple fold over even fresh whole blood. The solution given first (“PEG first”) opens up capillaries and thus makes subsequent hemoglobin carriers (blood or HBOC) much more efficient. It is less effective to add oxygen carrying capacity (whole blood or blood components) to shocked patients if their tissue capillaries are compressed closed by swollen cells in the tissue. This solution is designed to be administered to a shock victim first, i.e., before solutions 2) and (optionally) 3) below.
2) This new effective platform is improved by the safe addition of an HBOC (hemoglobin-based oxygen carrier) to restore oxygen carrying capacity in austere environments after massive loss of hemoglobin and without the need of field blood donors, as well as in specialized surgical or medical settings requiring high frequency of routine transfusions so as to limit risks of allogenic blood transfusions. The PEG module (described above) is administered first to open up capillary perfusion and then (second) small amounts of HBOC comprising L-Arginine are used to improve total oxygen carrying capacity and mitigate nitric oxide oxidation. This use of HBOC is safe and effective because of the present concomitant restoration of nitric oxide synthesis in the microcirculation using the natural NO substrate (L-Arginine) to protect the sensitive gut and other ischemic tissue beds from vasoconstriction due to the free hemoglobin (HBOC). This NO innovation is protective per se in shock (20) and allows safe use of HBOCs.
3) Third, coagulation and platelet function are restored with various options for hemostasis. These critical functions are lost in coagulopathy of trauma with hemorrhage of clotting factors and platelets, as well as due to hemodilution secondary to PEG-induced volume expansion and transient platelet thrombaesthenia secondary to direct competitive inhibition of fibrinogen binding to platelet IIb/IIIa receptors. This is reversed by administering to the shock victim a lyophilized fibrinogen concentrate solution or other potential preserved hemostasis alternatives such as lyophilized plasma, or preserved human or synthetic platelet products.

Novel administration systems, including a field kit, are also provided to administer all components easily by medics in the field in the order in which they need to be given for maximum effectiveness and appropriate administration with inter-module flushing.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this disclosure to provide an intravenous (IV) delivery device comprising: a first container containing a first solution comprising 18,000-100,000 Da PEG polymers and, optionally, 1,000 up to 18,000 Da PEG polymers in a first physiologically compatible liquid carrier; and a second container containing a second solution comprising a hemoglobin-based oxygen carrier (HBOC) in a second physiologically compatible liquid carrier; wherein the first and second containers are each configured to receive an IV drip, such as an IV drip chamber. In some aspects, the18,000-100,000 Da PEG polymers are 20,000 Da PEG polymers (PEG-20 k) and the 1,000 up to 18,000 Da PEG polymers are 8,000 Da PEG polymers (PEG 8 k). In other aspects, the second solution further comprises an agent that mitigates NO oxidation. In additional aspects, the agent that mitigates NO oxidation is L-arginine. In further aspects, the first and second containers are flexible IV bags. In yet further aspects, the first and second containers are attached via a liquid impermeable seam. In other aspects. The IV delivery device further comprises a container comprising physiological saline solution. In other aspects, the first and second physiologically compatible liquid carriers are the same or different. In additional aspects, the first and second containers contain from 25-500 mls of liquid, and the liquid volumes contained in the first and second containers are the same or different. In some aspects, the first solution comprises 250 ml of 200 mg/ml (20%) PEG-20 k and, optionally, 20 mg/ml (2%) PEG-8 k in the first physiologically compatible liquid carrier; and/or the second solution comprises 250 ml of 12 mg/ml HBOC and, optionally, 88.2 mg/ml L-arginine in the second physiologically compatible liquid carrier. In other aspects, the first and second physiologically compatible liquid carriers comprise one or more of NaCl, sodium lactate, KCl and CaCl₂. In additional aspects, the first and second physiologically compatible liquid carriers comprise 1.2 mg/ml NaCl, 0.62 mg/ml sodium lactate, 0.06 mg/ml KCl and 0.04 mg/ml of CaCl₂. In some aspects, the IV delivery device further comprises a third container containing a third solution comprising at least one hemostasis agent in a third physiologically compatible liquid carrier. In some aspects, the at least one hemostasis agent is lyophilized fibrinogen concentrate or lyophilized plasma. In further aspects, the third solution comprises, 100-250 ml of reconstituted fibrinogen (10-50 mg/ml) in the third physiologically compatible liquid carrier, and/or 100-250 ml of reconstituted lyophilized plasma in the third physiologically compatible liquid carrier.

The disclosure also provides a method of treating a subject suffering from blood loss, comprising i) administering to the subject a therapeutically effective amount of a first solution comprising 18,000-100,000 Da PEG polymers and, optionally, 1,000 up to 18,000 Da PEG polymers in a first physiologically compatible liquid carrier, and then ii) administering to the subject a therapeutically effective amount of a second solution comprising a hemoglobin-based oxygen carrier (HBOC). In some aspects, the 18,000-100,000 Da PEG polymers are 20,000 Da PEG polymers (PEG-20 k) and the 1,000 up to 18,000 Da PEG polymers are 8,000 Da PEG polymers (PEG 8 k). In additional aspects, the second solution further comprises an agent that mitigates NO oxidation. In yet further aspects, the agent that mitigates NO oxidation is L-arginine. In other aspects, the first solution comprises 250 ml of 200 mg/ml PEG-20 k and, optionally, 20 mg/ml PEG-8 k in the first physiologically compatible liquid carrier; and the second solution comprises 250 ml of 12 mg/ml HBOC and, optionally, 88.2 mg/ml L-arginine in the second physiologically compatible liquid carrier.

In other aspects, the method further comprises iii) administering to the subject a therapeutically effective amount of a third solution comprising at least one hemostasis agent, wherein step iii) of administering is performed after step ii) of administering. In some aspects, the at least one hemostasis agent is fibrinogen or reconstituted lyophilized plasma. In further aspects, the third solution comprises 100-250 ml of 10-50 mg/ml fibrinogen in the third physiologically compatible liquid carrier, and/or 100-250 ml of reconstituted lyophilized plasma in the third physiologically compatible liquid carrier. In some aspects, the third solution is administered after the first solution and before the second solution.

The disclosure also provides a kit comprising, the IV device as described above and an IV infusion set.

### BRIEF DESCRIPTION OF THE DRAWINGS

FIG. 1. Schematic representation of an administrative device of the disclosure.

FIG. 2. Nitric oxide (NO) synthesis by NOS uses L-arginine (A). In shock, asymmetric dimethyl arginine (ADMA) is produced that competes with L-Arg for NOS so NO drops (B1). Adding high amounts of L-Arg in shock overcomes the ADMA-induced NOS inhibition and restores NO synthesis in shock (C1). When HBOC is added during shock, further reductions in NO are caused by scavenging of formed NO by the HBOC (B2). Finally, L-arg add back overcomes this by driving a NOS rightward shift to synthesize more NO (C2).

FIG. 3. Anesthetized rats were administered 6.8 ml/kg HBOC-201 IV with a volume control of LR over 5 min and mean arterial blood pressure responses were observed (closed circle symbols). Blood pressure significantly rose over 30 minutes as the HBOC consumed local nitric oxide in the vessel wall and removed the natural vasorelaxation tone. However, the response to the same dose of HBOC was prevented (and even reversed) when 300 mg/kg of L-Arginine was co-administered with the HBOC (open squares). This strongly supports nitric oxide regeneration by L-Arg that offsets the loss by HBOC.

FIG. 4. A modified rodent hemorrhagic shock model that produces a DVO2 limiting oxygen debt following recovery. After rats reached the lactate target after arterial hemorrhage, as described previously, they were resuscitated with 6.8 ml/kg PEG-20 k IV solution. After 30 minutes when their pressure and lactates began to stabilize, an additional 10% estimated blood volume was slowly removed. The blood was centrifuged and the plasma was given back. The removed red blood cells in the pellet was sufficient to cause the rats to re-build lactate 12 hours after resuscitation and recovery from anesthesia. The debt builds through 36 hours and then begins to fall over the next 2 days. Survival in this model is 50% since half of the rats died after 24 hours.

FIG. 5. Capillary perfusion was measured by Orthogonal Polarization Spectral Imaging (OPSI) in the distal ileal mucosa in shocked swine. Values of capillary perfusion (PPV) were obtained before shock, after shock, and at various times after resuscitation with equal volumes (6.8 ml/kg) of 10% PEG-20 k IV Solution, Lactated Ringers vehicle, and fresh autologous whole blood. Values shown are mean+/−Standard error of the mean from 5-6 independent swine shock studies per group. These data clearly show the direct comparisons of PEG-20 k with whole blood where PEG-20 k solution with low oxygen carrying capacity (0.41 ml O2/dl) has a much higher capillary perfusion compared to whole blood that carries much more oxygen (21 ml O2/dl). Therefore, these data demonstrate the value of administration of PEG-20 k IV solution first during shock resuscitation followed by a hemoglobin-based oxygen carrier (HBOC) to maximize the positive attributes of both solutions when given in the proper order. PEG-20 k solution first opens capillaries to accommodate subsequently administered oxygen carriers, which maximizes oxygen transfer to ischemic tissues after shock. HBOCs like HBOC-201 are preferred oxygen carriers compared to whole blood because they are stable solutions that can be shelf stored without refrigeration for prehospital use in the field or for use in community hospital mass casualty situations where whole blood is not available in sufficient quantities.

### DETAILED DESCRIPTION

The present disclosure provides a stable complete bio-artificial blood substitute system that is used to safely and efficaciously replace blood volume, for example, in shock victims. The blood substitute is especially useful under austere or emergent conditions such as on the battlefield or during mass casualty events since the components can be stored at ambient temperature for extended periods of time. In particular, the system includes a field stable modular biosynthetic low volume blood substitute system comprising two to three sequentially ordered solutions for bridging patients until a blood transfusion is available. This is especially advantageous in settings where whole blood or blood products are not available or where cold storage is not realistic. In various aspects, the system is used as a bridge to transfusion in the field such as on the battlefield, during natural disasters, in the aftermath of mass shootings, etc. or anywhere when traumatic injury leads to dangerous blood loss in victims. However, the system is also useful in hospital settings, for example, where blood products cannot be used for religious beliefs of the patient or anytime there is an increased need for blood (e.g., high consuming operations such as cardiovascular procedures, trauma surgery, liver transplantation, etc.) or there is a significant lack of blood supply (e.g., as seen during the COVID-19 pandemic with decreased donations world-wide). This solution and device do not require refrigeration and are stable under harsh environmental conditions for months, so can be shelf stored or stored in the field for use.

The system is modular since the major components are not compatible in solution but are administered separately in a particular ordered sequence of two to three different solutions (modules, components) each of which performs a specific function. When the solutions are used in the correct order, the end result is functionally similar or superior to a transfusion of fresh whole autologous blood.

The system described herein (i.e., modular solutions delivered sequentially generally via a co-packaged IV-aid device) bridges patients to blood and blood product transfusion by (1) mimicking whole blood in oxygen-carrying capacity and coagulation potential and (2) promoting delivery of each of these components to the microcirculation in the tissues, which other solutions and even whole blood do not do well.

Finally, the system embodies a new device to ensure correct and safe administration of all of the components.

### The Modules

The three solution modules, which may be present in a single delivery device with the option of the third hemostasis component include:

### 1. A Perfusion Module:

Cells swell in response to ischemia and hypothermia, both of which occur with hemorrhagic hypotension. Cell swelling is primarily caused by failure of energy-dependent cell volume control mechanisms. Sodium pump (Na/K ATPase) function becomes impaired during shock, due to a lack of adequate ATP due to altered oxygenation and cellular energetics. As a result, sodium ions enter cells down electrochemical gradients. Na+ions recruit Cl— ions electrogenically, followed by passive water movement leading to cell swelling. Hydropic degeneration from energy failure damages membrane and mitochondrial structures, which may lead to cell death. Swelling of parenchymal and stromal cells can also compress local capillaries leading to further reductions in capillary flow and oxygen delivery causing a self-amplifying cycle of injury. Tissue and cell swelling during resuscitation can cause the “no reflow phenomenon” that limits positive resuscitation outcomes, even with hemoglobin rich blood and blood products. Essentially, no reflow is when there is ongoing microvascular ischemia despite macrovascular resuscitation due to closed capillaries. Additionally, low flow states, pro-inflammatory responses, and acid base disturbances in critical illness and shock can also lead to poor perfusion through red blood cell (RBC) rouleaux formation, which is a stacking of the RBCs in the capillaries. This trap further impedes flow, increases local blood viscosity, and propagates inflammatory cascades to further disrupt the microcirculation. Cell impermeant molecules can passively reverse this by osmotically holding water outside the cell. This water transfer prevents cell swelling, capillary compression, and no reflow.

A cell impermeant is a molecule that freely escapes the capillary space but cannot cross cell membranes, usually because the molecule is too large, too charged, or both. Cell impermeants are not necessarily colloids but colloids are cell impermeants. Typically, cell impermeants are saccharides with a mass of 200-700 daltons, anions like sulfate or phosphate, or charged saccharide species like gluconate or lactobionate. Ideal impermeants are also stable non-metabolizable species that remain osmotically active. These molecules are the most effective components of modern-day organ preservation solutions. They prevent cell swelling in cold-stored organs for transplantation. Since ischemia during shock and trauma leads to cell swelling injury through the same mechanisms (16, 21), these agents were useful for the development of the present systems and methods. Additionally, these agents are non-toxic, highly stable under extreme environmental conditions, work with very low fluid volume because they are highly soluble and are economical to produce. Thus, they are excellent candidates for LVR (low volume resuscitation) in severe shock.

Accordingly, the first module comprises a solution of the therapeutic PEG polymers that are cell impermeants in a physiologically acceptable carrier. This solution causes improved capillary perfusion and increases capillary perfusion pressure in the arterial system to accommodate good tissue capillary flow (see issued US patent U.S. Pat. No. 11,007,227, the complete contents of which are hereby incorporated by reference in entirety). This solution is administered first (i.e., “PEG first”).

Polyethylene Glycol (PEG): Repeating units of ethylene glycol (polyethylene glycol-PEG) range in size from e.g., 100-8,000,000 daltons. Polymers above 1000 are nontoxic to animals and sizes above 500 generally act as impermeants. Polymers of PEG between 18,000-80,000 are still cell impermeants but their variable permeability to the capillary gives them some oncotic strength. Polymers of 80-100,000 Da and greater likely possess mostly colloidal properties. Additionally, PEG polymers are extremely hydrophilic and avidly attract water shells around the molecule. PEG-20 k (20,000 MW) was found to have both impermeant and colloidal properties based on its oncotic reflection coefficient, as it distributes about ⅓ outside of the capillary into the interstitial space (impermeant actions) and about ⅔ inside capillaries where it exerts oncotic actions. This means for every 2 molecules of PEG-20 k that stays in the capillary space, 1 exits and enters the interstitial space. This unique group of intermediate sized PEGs (including PEG-20 k) thus represents true hybrid molecules possessing both impermeant and oncotic actions due to the unique size, molecular radius, and differential distribution in the microcirculatory volume compartments (i.e. its intermediate osmotic reflection coefficient). We have determined that these characteristics produces profound resuscitation effects in shock and this agent has been incorporated into the present systems. The strong effect seen with this intermediate PEG range is caused by the decompression of the microcirculation, where PEG is able to augment water transfer to reload the capillaries with volume to enhance driving pressure for capillary flow. This is due to two main phenomena: 1) hybrid nature with both impermeant and partial oncotic properties, and 2) highly hydrophilic nature to attract water molecules. These characteristics allow efficient capillary perfusion and transfer of oxygen into the tissues even during low volume or permissively hypotensive states.

Separately, smaller PEG polymers of size 1000-18000 Da are also incorporated in the solution to provide two important actions: 1) to attenuate erythrocyte sedimentation rates seen with RBC rouleaux formation due to shock or critical illness, or as seen with addition of the intermediate sized PEGs to whole blood in vitro, and 2) to provide short-term immunocamouflage of proinflammatory cascades from an activated immune system due to shock and critical illness especially early after resuscitation. Immunocamouflage is a non-specific effect of PEG polymers due to surface passivation of blood cells by the polymers, thereby “coating” surfaces and cloaking them from activated factors from injured tissue. PEG sizes used are readily available from chemical supply companies.

In the present disclosure, a first solution comprising PEG polymers in a physiologically acceptable carrier is administered to a subject who has experienced severe blood loss or clinically requires transfusion. The combination of intermediate and lower PEG weights creates an ideal composition to enhance therapeutic perfusion effects on local capillary networks. Thus, embodiments of the disclosure provide a composition comprising PEG with a molecular weight of 18,000-100,000 Da, e.g. 18,000-40,000 Da, e.g. 20,000-35,000 Da, e.g. 18,000 Da, 20,000 Da, 25,000 Da, 30,000 Da, 35,000 Da, or 40,000 Da at a concentration of 5-30% by weight per volume (w/v), e.g. 5-20%, 10-30%, or 10-20% w/v, g/L total solution. The composition further comprises PEG with a molecular weight of 1,000-18,000, e.g. 2,000-8,000 Da, e.g. 2,000, 3,000, 4,000, 5,000, 6,000, 7,000, 8,000, 9,000, 10,000, 11,000, 12,000, 13,000, 14,000, 15,000, 16,000, 17,000, or 18,000 Da, e.g. 6,000 Da at a concentration of 1-30%, e.g. 1-20% or 1-10% w/v, g/L total solution.

The PEG is dissolved in a physiologically compatible aqueous carrier. In some aspects, the carrier is saline and typically includes NaCl and optionally one or more additional physiologically compatible salts such as sodium lactate, potassium chloride and/or calcium chloride (e.g., dissolved in sterile, distilled water). Any physiologically compatible saltwater base may be used.

Sodium ions are generally present in the range of from about 100 to 140 mM per 1000 ml of solution, i.e., about 100, 110, 120, 130 or 140 mM per 1000 ml. A typical solution contains about 130 mM of sodium ions per 1000 ml of solution.

Lactate is generally present in the range of from about 20 to 40 mM per 1000 ml of solution, i.e., about 20, 21, 22, 23, 24, 25, 26, 27, 28, 29, 30, 31, 32, 33, 34, 35, 36, 37, 38, 39 or 40 mM per 1000 ml. A typical solution contains about 28 mM of lactate per 1000 ml of solution.

Potassium ions are generally present in the range of from about 1 to 5 mM per 1000 ml of solution, i.e., about 1, 2, 3, 4, or 5 mM per 1000 ml. A typical solution contains about 4 mM of potassium per 1000 ml of solution.

Calcium is generally present in the range of from about 1 to 3 mM per 1000 ml of solution, i.e., about 1, 2, or 3 mM per 1000 ml. A typical solution contains about 2.7 mM of calcium per 1000 ml of solution.

Chloride ions are generally present in the range of 90-120 mM per 1000 ml of solution, i.e., about 90,100, 110, or 120 mM per 1000 ml of solution. A typical solution contains about 109 mM chloride ion per 1000 ml of solution.

The pH of the carrier is typically in the range of from about 6.0 to about 7.0, for example, about 6.0, 6.1, 6.2, 6.3, 6.4, 6.5, 6.6, 6.7, 6.8, 6.9, or 7.0. In some aspects, the pH is about 6.5.

This solution is typically sterilized before storage for use, has a long shelf life and is stable at ambient temperatures, even extreme temperatures. There is no need for refrigeration.

In some aspects, the carrier is Lactated Ringer's solution. This carrier is readily commercially available from biological supply companies and contains: 60 mg of sodium chloride, 31 mg of sodium lactate, 3 mg of potassium chloride, and 2 mg of calcium chloride per 50 ml of solution (pH 6.5).

Generally, about 3.4 ml/kg body weight of this first solution (at 20% weight to volume of PEG-20 k) is administered to a blood loss/shock victim. For example, about a 3.0, 3.1, 3.2, 3.3, 3.4, 3.5, 3.6, 3.7, 3.8, 3.9, or 4.0 ml/kg body weight dose is administered. Typically, the dose is 3.4 ml/kg body weight for an adult or for a child.

Accordingly, when packaged into the deployable products disclosed herein, the container which contains this first solution (such as an IV bag) will generally contain one dose of solution, e.g., about 200-300 ml of solution, but may be expanded to about 25-500 ml as needed for patient and clinical scenario.

The first solution is administered as soon as possible after a shock victim is identified. Administration is generally by IV and the rate of administration (especially in the field i.e., away from a hospital setting, or in an underequipped hospital) is as rapid as is safely possible, for example, the contents of the bag containing the first solution are delivered completely intravenously in 3-5 minutes.

### 2. An Oxygen Carrier Module:

The perfusion module (above) is not able to carry oxygen except that which can be dissolved in the water component of the solution. Even though the increased oxygen transfer in the microcirculation caused by PEG perfusion solution is enough by itself to repay oxygen debt in shock and ischemia, the lack of oxygen carrying capacity limits its use. Therefore, a second module comprising a hemoglobin-based oxygen carrier (HBOC) is administered to add oxygen capacity.

Commercially available HBOCs that can be utilized in the practice of the present methods include but are not limited to: glutaraldehyde-polymerized bovine Hb (HBOC-201), glutaraldehyde-polymerized human Hb (Polyheme), maleimide-pegylated human Hb (MP4OX), or α-α diaspirin crosslinked human Hb (HemAssist). It is vital to select an HBOC that is non-toxic e.g., that is large enough to not cross the capillary space and so avoid problems with renal toxicity due to oxidative stress.

The HBOC is generally dissolved or suspended in a physiologically compatible carrier, generally an aqueous saline solution such as those described above for the PEG solution (first module). The HBOC is typically present in an amount of about 12 mg/dl.

In some aspects, the hemoglobin-based oxygen carrier is HBOC-201. HBOC-201 (Hemopure®) is a synthetic, second-generation glutaraldehyde-polymer of bovine hemoglobin, i.e., a purified, cross-linked and polymerized acellular bovine hemoglobin in a modified lactated Ringer's solution. HBOC-201 can serve as an “oxygen bridge” to maintain oxygen carrying capacity while transfusion products are unavailable. HBOC-201 is safe, stable, and has spectral characteristics almost identical to human hemoglobin, which makes the solution compatible with clinical human hemoglobin blood gas analyzers. HBOC-201 advantageously does not require blood compatibility with the recipient.

HBOC-201 has been successfully used in emergency settings for polytrauma, hypovolemia-induced cardiac arrest, and hemorrhagic shock.

An additional component for the HBOC module is the amino acid L-Arginine (L-Arg). Local nitric oxide (NO) synthesis is critical for survival from hemorrhagic shock. The use of HBOCs exacerbates nitric oxide dependency because HBOCs and naked hemoglobin are known scavengers of NO through a rapid oxidation mechanism. HBOCs in general are plagued with a nitric oxide scavenging effect that increases blood pressure secondarily to limiting tissue perfusion from vasoconstriction of precapillary sphincters. The exact chemical mechanism is unclear but may involve oxidation of NO to nitrite and formation of met-hemoglobin. Thus, a nitric oxide mitigation strategy is used with the HBOC.

The deleterious NO scavenging effect is mitigated in the present HBOC solution by the addition of L-Arg IV to the HBOC solution. Generally, L-arginine is added in an amount ranging from about 100 to 500 mg/kg, such as about 100, 150, 200, 250, 300, 350, 400, 450 or 500 mg·kg. In some aspects, 300 mg/kg of L-Arg is used, which is achieved with an L-Arginine concentration of 88.2 mg/ml HBOC-201 when the HBOC is dosed at 3.4 ml/kg, which is a typical recommended dose. These amounts of L-Arg are chemically compatible with e.g., HBOC-201 solution in the second module.

This hemoglobin-based oxygen carrier/L-arginine solution is administered second, after the first perfusion module. Further, since the two solutions are incompatible at their formulated concentrations, they also must not be mixed in a delivery device. For example, a saline rinse is required between the two to wash out IV tubing that has been used to deliver the first modular solution. Alternatively, each module may be delivered through a different, separate tube.

Generally, about 3.4 ml/kg body weight of the second solution is administered to a blood loss/shock victim. For example, about a 3.0, 3.1, 3.2, 3.3, 3.4, 3.5, 3.6, 3.7, 3.8, 3.9, or 4.0 ml/kg body weight dose is administered. Typically, the dose is 3.4 ml/kg body weight for an adult or for a child.

Accordingly, when packaged into the deployable products disclosed herein, the container which contains this second solution (such as an IV bag) will generally contain one dose of solution, e.g., about 250 mls of solution.

The second solution is administered as soon as possible after a shock victim is identified but only after the first solution, described above, is administered. Generally, the second solution is administered within 6 hours of the first solution, e.g. at least within about 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40 or 30 minutes, such as within about 30, 25, 20, 15, 10 or 5 minutes or less, (4, 3, 2, or 1 minute) after the first solution is administered. Administration is generally by IV and the rate of administration (especially in the field i.e., away from a hospital setting, or in an underequipped hospital) is as rapid as is safely possible, for example, the second solution is administered IV in about 3-5 minutes.

In some aspects, the second (oxygen carrying capacity) HBOC solution may be exchanged for a perfluorocarbon-based solution to delivery oxygen that would not require the nitric oxide mitigation strategy, but could require an alternate mitigation strategy for its own side effects such as complement activation.

The pH of the carrier is typically in the range of from about 6.0 to about 7.5, for example, about 6.0, 6.1, 6.2, 6.3, 6.4, 6.5, 6.6, 6.7, 6.8, 6.9, 7.0, 7.1, 7.2, 7.3, 7.4, or 7.5. In some aspects, the pH is about 6.5.

This second solution is typically sterilized before storage for use, has a long shelf life and is stable at ambient temperatures, even extreme temperatures.

### 3. Optional Coagulation Module:

Fibrinogen is a plasma glycoprotein with a molecular weight of 340 kDa; it is synthesized by the liver. Fibrinogen is a soluble protein that is naturally present in blood plasma, and from which fibrin is produced by the action of the enzyme thrombin. Severe blood loss results in loss of all coagulation factors, including fibrinogen, which is necessary for formation of fibrin nets and for coagulation of the liquid blood component. Restoring this “universal” coagulation factor helps restore coagulation and platelet aggregation, since fibrinogen also plays a role in cross linking platelets and serves to strengthen existing clots.

Fibrinogen treatment leads to increased clot firmness in dilutional coagulopathy after fluid resuscitation. Locally, this increased clot strength and thrombus formation in local injured vessels is associated with increased patient survival when combined in the correct ratio with packed cell components. Presumably, this is due to replacement of lost fibrinogen from blood loss and from decreased fibrinogen concentration in dilutional coagulopathy 25following non-plasma resuscitation. These properties of fibrinogen are utilized in the present systems and provide added value by reversing some of the known mild inhibitory effects of PEG-20 k on platelet interactions with fibrinogen, which would otherwise form platelet aggregates that contribute to clot size and firmness. The mechanism of the platelet effect by PEG-20 k is believed to be due to transient interference of fibrinogen binding to platelet fibrinogen receptors or due to non-specific interactions of intermediate sized PEGs (e.g. PEG-20 k) with cell components of the coagulation system, such as what is seen with immunocamoflague effect described above. Administration of fibrinogen overcomes this slightly inhibitory competition by causing a mass action shift.

Freeze-dried plasma is an alternate hemostatic module option as opposed to fibrinogen alone, as it includes fibrinogen in addition to plasma proteins (e.g. albumin), physiologic anticoagulants (e.g. protein C, protein S, antithrombin, tissue factor pathway inhibitor), all coagulation factors (i.e. II, VII, VIII, IX, X, XI, and vWF), as well as electrolytes, fats, and sugars. Basically, plasma without the aqueous component. The benefits of freeze-dried over fresh frozen plasma are intended for the field in a pre-hospital setting due to storage stability. Currently available lyophilized products generally perform the same as fresh frozen plasma in patient outcomes and in restoring coagulation in many trauma trials with similar side effect profiles (13, 14).

Accordingly, the third solution that is (optionally) administered to a subject that is treated by the present modular blood substitute systems and methods is a hemostasis solution that may include lyophilized (freeze-dried) fibrinogen concentrate or lyophilized plasma.

Subjects who are fibrinogen recipients generally exhibit hypofibrinogenemia, defined by a decreased level of normal fibrinogen between 0.5-1.5 g/L, the lower limit of the normal range being usually 1.5 g/L. Afibrinogenemia is the absence of plasma fibrinogen. In field trauma situations, the subject will not usually be tested to determine fibrinogen levels. However, in a hospital setting, such testing may be available.

The type of fibrinogen may vary. Options include fibrinogen cryoprecipitate, which typically contains about 15 g/L fibrinogen in addition to coagulation factors VIII, XIII, and vWF. However, fibrinogen concentrate produced from pooled human plasma (e.g., using the Cohn/Oncley cryoprecipitation procedure) is generally preferred. In these products, the concentration of fibrinogen is standardized and the product is generally stored as a lyophilized powder at room temperature and can be reconstituted rapidly with sterile water or an aqueous based solvent/carrier, such as lactated Ringers. Infusion volumes can be kept low, allowing for rapid administration without delays for thawing or cross-matching. Further, viral inactivation steps by solvent/detergent exposure or pasteurization are routinely included in the manufacturing process for fibrinogen concentrate, as well as for lyophilized plasma, thus minimizing the risk of viral transmission.

Four fibrinogen concentrates are currently available: Haemocomplettan® (CSL Behring, Marburg, Germany), FIBRINOGENE T1 and Clottagen (LFB, Les Ulis, France), Fibrinogen HT (Benesis, Osaka, Japan) and FibroRAAS® (Shangai RAAS, Shangai, China). The most widely used is Haemocomplettan® (commercialized in the USA as RiaSTAP®) 18, a human pasteurized, highly purified, plasma-derived fibrinogen concentrate.

Three lyophilized plasma (LP) products are currently available that have undergone trials in field trauma: (1) French Lyophilized Plasma (FLYP), produced by the French Military Blood Institute (Centre de Transfusion Sanguine des Armees [CTSA]); (2) LyoPlas N-w, produced by the German Red Cross; (3) Bioplasma FDP, produced by National Bioproducts Institute, Pinetown, South Africa (13).

In some aspects, the freeze-dried fibrinogen or plasma is a commercially available product as listed above. These products are lyophilized and readily reconstituted as a fibrinogen concentrate or plasma solution suitable for use at the third modular solution in the present systems and methods.

Whatever the source, a dose of about 4,900 mg of fibrinogen is typically used for a 70 kg adult (70 mg/kg). Thus, a solution of 32.7 mg/ml of fibrinogen concentrate in sterile water is typically used in the present systems and methods and dosed at 2.1 ml/kg body weight or about 150 ml for a 70 kg adult.

For lyophilized plasma, the minimal volume of reconstitution that is safe for infusion is known to be about 50% of the original plasma volume, which is typically 200-250 ml for standard units of plasma like fresh frozen plasma. Both French and German products use 200 ml of sterile water to rehydrate the product within 3-10 minutes. This volume (200 ml) is given at a rate of 5-10 ml/min or as fast as tolerated. Thus, a low volume option may provide 100 ml volume.

Generally, about 100-500 ml of this third solution is administered to a blood loss/shock victim. For example, about a 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500-ml dose is administered. Typically, the dose is 150 ml for an adult and 50 ml for a child based on the 70 mg/kg dose for fibrinogen concentrate when the patient's fibrinogen level is not known.

Accordingly, when packaged into the deployable products disclosed herein, the container which contains this third solution (such as an IV bag) will generally contain one dose of solution, e.g., about 100-500 mls of solution.

The third solution is also administered as soon as possible after a shock victim is identified, but after both the first and second solutions, described above, are administered.

Generally, the third solution is administered within six hours of the second solution, e.g. at least within about 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40 or 30 minutes, such as within about 30, 25, 20, 15, 10 or 5 minutes or less, (4, 3, 2, or 1 minute) after the second solution is administered. Administration of the fibrinogen or plasma solution is generally by IV (intravenous) route and the rate of administration (especially in the field i.e., away from a hospital setting, or in an underequipped hospital) is at about 5-10 ml/min or as fast as tolerated.

In other aspects, the third (hemostasis) modular solution is administered after the first (PEG) modular solution, but before the second (HBOC) solution, i.e., in between the PEG and HBOC solutions. This hemostasis solution is generally administered within six hours of the first (PEG) solution, e.g. at least within about 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40 or 30 minutes, such as within about 30, 25, 20, 15, 10 or 5 minutes or less, (4, 3, 2, or 1 minute) after the second solution is administered. Administration is as described above. After both the PEG and hemostasis solutions have been administered, in some cases the HBOC (oxygen carrying capacity) solution may then be administered within six hours of the hemostasis solution, e.g. within about 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40 or 30 minutes, such as within about 30, 25, 20, 15, 10 or 5 minutes or less, (4, 3, 2, or 1 minute) after the second solution is administered. Administration is as described above.

The pH of the carrier is typically in the range of from about 6.0 to about 7.5, for example, about 6.0, 6.1, 6.2, 6.3, 6.4, 6.5, 6.6, 6.7, 6.8, 6.9, 7.0, 7.1, 7.2, 7.3, 7.4, or 7.5. In some aspects, the pH is about 6.5.

This third solution is also typically sterilized before storage for use, has a long shelf life and is stable at ambient temperatures, even extreme temperatures.

This solution is delivered third after the HBOC/L-Arg solution. A saline wash of the tubing may be conducted between administration of the second and third solutions.

### Devices and Kits

In some aspects, the modular solutions are delivered by a custom type of flexible multi-compartment IV bag (e.g. a Viaflex or other flexible IV bag) that is designed to provide physical separation of each of the three components before sequential delivery of each. An exemplary multi-compartment IV bag with walls or seams between the compartments is taught, for example, in WO2017005265A1. Similarly, issued U.S. Pat. Nos. 4,925,444 and 4,637,817 discuss and provide multicompartment IV bags.

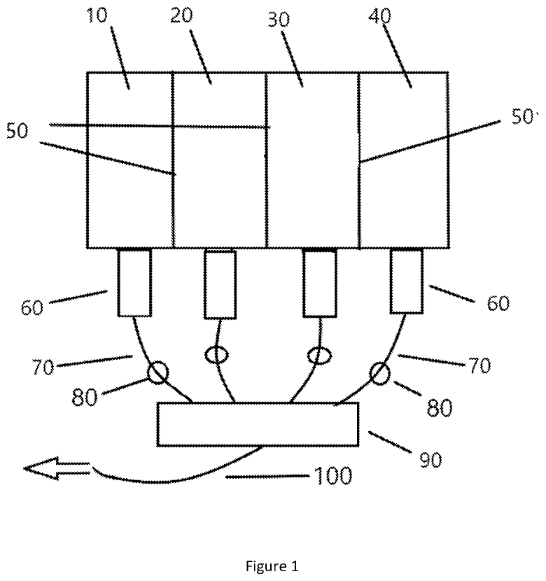

A schematic illustration of a multicompartment IV bag is shown in FIG. 1. As can be see, the device comprises compartments 10 and 20 and optional compartments 30 and 40. Compartments 10 and 20 (and optional compartments 30 and 40, if present) are separated by wall or seam 50 to prevent mixing, between the compartments, of the liquid solution which each contains, i.e. the seam is impermeable to liquid. Each bag comprises a port (not shown) which is pierceable by a spike (not shown) of drip chamber 60, which leads to IV tubing line 70. Clamp 80 (which may be a roller clamp, a slide clamp, etc.) regulates the rate of fluid delivery to the patient. These components are generally part of what is referred to in the art as an “infusion set”. Generally, a “macroset” is used to deliver about 20 drops per minute, or about 100 mL per hour. However, microsets may also be utilized, e.g. for children.

In some aspects, line 70 goes directly to a blood vessel of a subject via a cannula and/or needle. In other aspects, line 70 goes to multiple liquid port connector for 90. Examples of such connectors are described, for example, in issued U.S. Pat. No. 10,549,084 and US patent application US20200155823, the complete contents of each of which is hereby incorporated by reference in entirety. While such connectors may be designed for use with a pump, they are also adaptable to field use where the IV flow is driven by gravity.

Still with reference to FIG. 1, line 100 directs the flow of a solution to a blood vessel of the subject that is receiving the IV solutions (the arrow shows the direction of flow), e.g. to a vein or artery, typically a vein, via a cannula or needle inserted directly therein (not shown). Those of skill in the art will recognize that this is a generic depiction of a device and that many other arrangements for the delivery of IV fluids may be possible.

Generally, compartments 10 and 20 contain solutions 1 and 2 respectively, as described herein. Optional compartment 3 contains, for example, optional solution 3 or saline if optional solution 3 is not included. Optional compartment 4 contains, for example, saline for saline rinses of the line between solutions. Alternatively, saline may be provided in a separate container and accessed as needed during administration.

Other components may be built into the device and/or the components thereof. For example, a luer lock collar, a vent, a waste port for egress of the saline wash, etc.

Further, the device is not limited to 2, 3 or 4 compartments. Additional compartments may contain, for example, other medicaments such as pain medication, antibiotics, nutrients, etc.

The sizes of each compartment in a device may or may not be the same. For example, a compartment for saline may be smaller than a compartment for solution 1, 2, or 3, as less saline may be needed.

Such devices may be provided as a kit. In the kit, the multi-compartment IV bag may be folded to conserve space, packed (e.g., rolled) around a wheel for storage, or packaged in some other efficient means which retains the integrity of the IV bags but is readily deployable.

Other possible elements of such a kit include but are not limited to: additional IV tubing, various clips and ports, a tourniquet, needles, alcohol swabs, labels, medical adhesive, surgical gloves, portable micropump, etc. Needles will typically be relatively large, e.g., 14 gauge is the largest and it is usually used to correct symptoms of shock and trauma. However, smaller sizes e.g., 18-20 may be used on smaller-sized adults or adolescents, while size 22 may be used for pediatric patients (such as infants, toddlers, and young children) or geriatric patients.

### Methods

This disclosure also provides methods of treating blood loss and/or shock in a subject in need thereof by administering, in order: a first solution comprising PEG polymers in a first physiologically compatible liquid carrier; then a second solution comprising a hemoglobin-based oxygen carrier (HBOC) and L-arginine in a second physiologically compatible liquid carrier; then a third solution comprising lyophilized fibrinogen concentrate or lyophilized plasma in a third physiologically compatible liquid carrier. The physiological carriers for the three solutions may be the same or different.

Those of skill in the art are well-acquainted with administering medicaments to a subject intravenously. The IV bag is prepared and a macroset or microset line is inserted into the port thereof while the valve flow is off. Generally, IV bags should be hung above the level of the patient's heart. The flow control is opened to let fluid (e.g. saline) run through the tubing and eliminate air bubbles (i.e. priming the line) and keeping the drip chamber about half filled. A tourniquet is tied directly above the location where the needle/cannula will be inserted. A cannula is inserted and secured, typically together with a needle, the needle is withdrawn, the cannula is secured and stays in place and the IV tubing fed into the cannula, making sure all connections are secured. The line is slowly opened to allow the desired flow rate (usually with normal saline first) and then the system is switched to administered other medications, namely solutions 1 and 2 as described herein (“piggyback”), then optionally solution 3, and any other medications that are needed, e.g. pain medications, antibiotics, etc. In a field setting, the IV flow rate is regulated manually, e.g. by counting the drops per minute, and adjusting until the proper rate is attained. However, some roller knobs have settings to particular rates, and IV machines in hospitals are typically set via digital input.

As indicated elsewhere herein, the present devices and solutions are stable at ambient temperature. Thus, they store well for long periods of time, do not require refrigeration or thawing, and are ideal for use in emergency situations where formal hospital care is not available. For example, they can be deployed on the battlefield, in war zones, in the aftermath of a natural or man-made disaster such as a bomb blast or mass shooting, or in any situation where rapid treatment of patients who have experienced severe blood loss and/or shock, e.g. hemorrhagic shock. However, these devices and solutions may also be advantageously used in formal patient care settings, e.g. in a hospital operating room when surgeries requiring copious amounts of blood are lost by the patient and must be replaced (e.g., transplant surgery, heart surgery, amputations, etc.) especially if there is a shortage of blood of the patient's blood type. The use of these solutions provides a respite while matching whole blood is obtained.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. Nitric Oxide Mitigation With HBOC

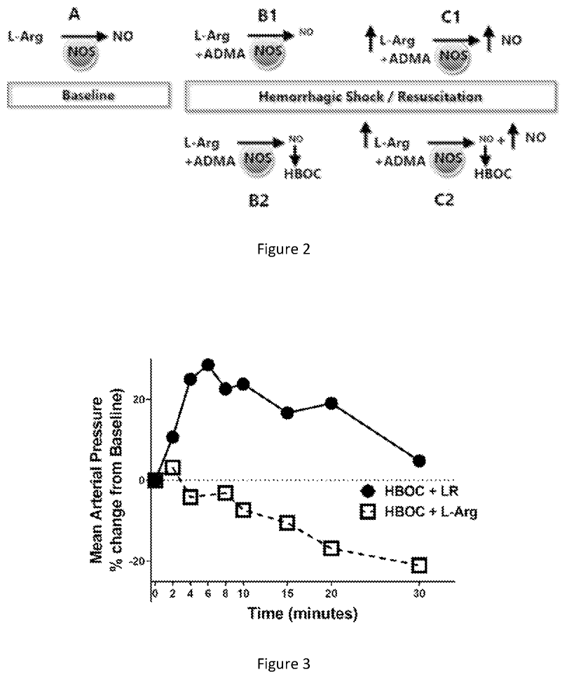

HBOCs are acellular hemoglobin molecules used to carry oxygen in bioartificial blood products. However, naked hemoglobin molecules scavenge local nitric oxide (NO) from the endothelium by oxidation at their heme redox centers. The reduced NO available in the microcirculation typically causes a 30% rise in systemic blood pressure and reduced capillary perfusion. This is not a critical problem until the tissue becomes ischemic and accumulates an oxygen debt. Loss of NO at reperfusion after ischemia or shock then critically limits tissue perfusion, oxygen delivery to starved tissues, and limits debt repayment. Our lab has demonstrated how crucial this mechanism is in the pathophysiology of hemorrhagic shock since replenishing local NO at resuscitation critically restores local perfusion to bowel mucosa and significantly improves outcomes. Conversely, blocking NO synthesis at resuscitation produces devastating results on survival outcomes and bowel architecture. Our lab also demonstrated that loading the microcirculation with L-Arginine was able to provide the needed NO synthesis, presumably by a rightward mass action shift in the Mchaelis-Menton enzyme kinetics of the local nitric oxide synthetase system. Based on these previous findings from our lab (20), L-Arginine loading is used in the present system to safely administer HBOC in shock as a mitigation strategy for NO depletion by HBOC. A cartoon showing these mechanisms and strategy for safe HBOC use in shock is shown in FIG. 2.

To further support that large amounts of infused L-arginine activates nitric oxide synthesis from eNOS in the wall of the blood vessel, we reversed the vasoconstrictor response of IV HBOC-201 administration in rats by coadministering L-Arginine. This is shown in FIG. 3.

These results show that administration of a dose of HBOC-201 (6.8 ml/kg) to anesthetized adult rats produced an immediate hypertension that persisted for 30 minutes. This hypertension is believed due to the local scavenging of nitric oxide in the vessel wall. As the normal vasodilator tone produced by basal nitric oxide is removed by the HBOC hemoglobin, the vasoconstrictor tone now prevails and the blood pressure rises secondary to vasoconstriction of the resistance vessels. The same dose of HBOC-201 combined with 300 mg/kg, L-Arg not only did not produced vasoconstriction but caused vasodilatation that caused the blood pressure to drop over the same period. These data suggest that L-Arg administration at these doses causes nitric oxide synthesis in the vessel wall that overcomes the loss from HBOC. Since vasodilation was observed after both HBOC and L-Arg, we conclude the net production of nitric oxide from L-Arg exceeded baseline levels, even with HBOC present, to relax the resistance vessels and produce a drop in central blood pressure. That this effect is nitric oxide dependent is supported by its reversal with the nitric oxide synthetase inhibitor L-NAME (22).

### Example 2. DVO2 Dependent Shock Model

A major goal of this research is to reconstitute chemical oxygen carrying capacity after resuscitation with PEG-20 k IV solution in severe hemorrhagic shock. The need to replace lost hemoglobin after PEG-20 k resuscitation is two-fold; 1) Severe bleeding leads to lost oxygen carrying capacity (RBCs), and 2) Rapid volume expansion after PEG-20 k resuscitation, while beneficial for restoring tissue perfusion, dilutes already low hemoglobin in the circulation. For animal models, the PEG-20 k IV solution resuscitation works so well at restoring oxygen transfer through efficient capillary blood flow, even in lethal hemorrhage states, that additional oxygen carrying capacity is not needed after the first post-operative recovery day. This is based on normal plasma lactate values. While this works for young healthy animals, human shock, where oxygen debt would be expected to rise without red blood cell transfusions, is more complex. Therefore, adjustments were made to the rodent shock model so that their oxygen delivery is slightly worse after resuscitation to precipitate a metabolic oxygen delivery problem that can be measured and “fixed” by the present methods. In contrast, for the swine model, we discovered that they did begin to build oxygen debt after 24 hours from recovery, similar to humans.

Therefore, we added an extra bleed in the rodent shock model to simulate re-bleeding in an uncontrolled shock state that causes some additional loss of hemoglobin on the day of resuscitation. This resulted in building of significant oxygen debt later and established a positive control to use in treating severe post resuscitation anemia and oxygen debt re-accumulation. In the rodent model, all experimental procedures were as previously described (16) except 30-60 minutes after low volume resuscitation, an additional arterial blood loss of 10% of the estimated blood volume was removed. The removed blood was centrifuged and the plasma was immediately given back to the rat. This selectively depleted the rat of additional oxygen carrying capacity (RBCs) at a safe period following resuscitation and caused significant downstream accumulation of oxygen debt after about 36 hours post resuscitation and anesthesia recovery.

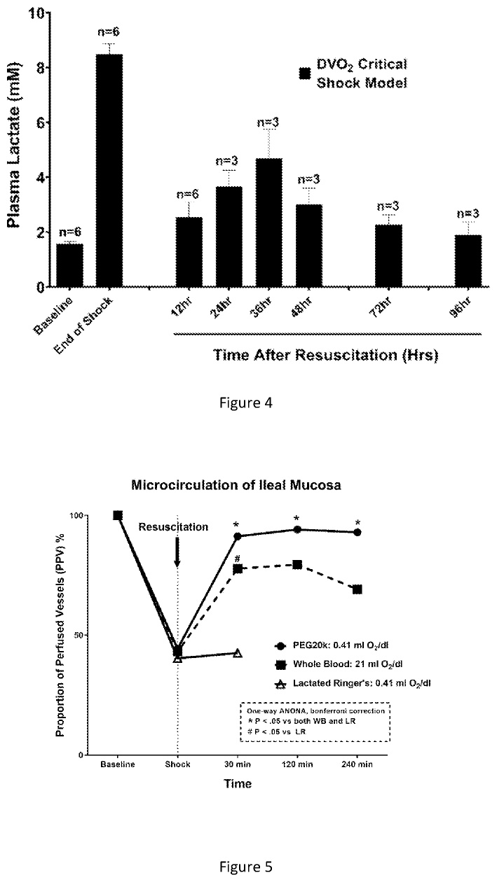

The results of this modified rodent shock model are shown in FIG. 4 and suggest the importance of supplementing the PEG-20 k low volume resuscitation solution with an oxygen carrier like field stable HBOC-201.

### Example 3

### Part 1. PEG-20 k and Delayed HBOC

Studies are conducted in the proven rodent model of lethal hemorrhagic shock and low volume resuscitation to demonstrate how timing of HBOC-201 following PEG-20 k IV solution can prevent any deleterious effects from NO scavenging while safely replenishing the vascular space with oxygen carrying capacity for improved long-term outcomes. Rats are bled to 35 mmHg and held there until lactate reaches 9-10 mM, at which time, LVR solutions are given to resuscitate them. Thirty minutes after resuscitation, another 10% blood volume is removed and the plasma component is immediately separated and given back. A loop of distal ileum is exposed and a 1 cm enterotomy is made along the anti-mesenteric border to access the mucosal surface for hourly recordings of capillary blood flow by Orthogonal Polarization Spectral Imaging (OPSI) and for biopsies of bowel mucosa at various times before. The biopsies are used for histological analysis and for nitric oxide metabolism studies ex-vivo in a tissue culture system. The experimental groups are listed in Table 1.

TABLE 1

| | | Lag Time |
|---|---|---|
| Group | LVR Solution | (hours) |
| 1 | Lactated Ringers (LR) solution (6.8 ml/kg) | n/a |
| 2 | 10% PEG-20k (6.8 ml/kg) | n/a |
| 3 | 20% PEG-20k (3.4 ml/kg) + whole | n/a |
| | blood (3.4 ml/kg) | |
| 4 | 20% PEG-20k (3.4 ml/kg) + HBOC | 0 |
| | 201 (3.4 ml/kg) | |
| 5 | 20% PEG-20k (3.4 ml/kg) + 6% | 0 |
| | albumin (3.4 ml/kg) | |
| 6 | 20% PEG-20k (3.4 ml/kg) + HBOC | 2 |
| | 201 (3.4 ml/kg) | |
| 7 | 20% PEG-20k (3.4 ml/kg) + 6% | 2 |
| | albumin (3.4 ml/kg) | |
| 8 | 20% PEG-20k (3.4 ml/kg) + HBOC | 4 |
| | 201 (3.4 ml/kg) | |
| 9 | 20% PEG-20k (3.4 ml/kg) + 6% | 4 |
| | albumin (3.4 ml/kg) | |

The albumin solution is a control for both the protein and oncotic effects of the HBOC per se and for the delayed volume administration at 2 and 4 hours after the initial PEG-20 k resuscitation. The total volume infusions for these studies is maintained at 6.8 ml/kg to remain in the low volume range (10% estimated blood volume). All surgical preparations are sterile so any animals surviving after 5 hours from resuscitation can be recovered the next day. N=10 rats per group.

### Part 2. PEG-20 k and Nitric Oxide Modified HBOC

Studies were designed to test how nitric oxide substrate loading before HBOC prevents or attenuates the NO scavenging effects of the HBOC on physiological, biochemical, and survival outcomes. The salutary effects of L-Arg in severe shock has been demonstrated previously to counteract the NOS inhibition due to endogenously produced asymmetric dimethyl arginine (ADME), which are commonly released in shock and critical illness. In rat experiments, we have administered HBOC in non-shocked rats either with L-Arginine together with HBOC or with a similar volume of L-Arg (control experiment). L-Arginine mitigated the hypertensive effect observed after HBOC alone (a phenomenon that is due to NO inactivation by the HBOC).

Any modifying effects of L-Arg on the HBOC groups are probed further using substrate level (D-Arg) and enzyme level (L-NAME) inhibitors of nitric oxide synthesis to confirm that L-Arg is working by a NO mechanism. The experimental groups are listed in Table 2.

TABLE 2

| | | Nitric Oxide |
|---|---|---|
| Group | LVR Solution | Variable |
| 1 | 10% PEG-20k (6.8 ml/kg) + | Vehicle control |
| | 0.3 ml LR | |
| 2 | 10% PEG-20k (6.8 ml/kg) + | L-Arg (300 mg/kg) |
| | L-Arg + 0.3 ml LR | |
| 3 | 10% PEG-20k (6.8 ml/kg) + | D-Arg (300 mg/kg) |
| | D-Arg + 0.3 ml LR | |
| 4 | 10% PEG-20k (6.8 ml/kg) + | L-Arg + L-NAME |
| | L-Arg + L-NAME | (10 mg/kg) |
| | in 0.3 ml LR | |
| 5 | 20% PEG-20k (3.4 ml/kg) + | Vehicle control |
| | HBOC-201 (3.4 | |
| | ml/kg) + 0.3 ml LR | |
| 6 | 20% PEG-20k (3.4 ml/kg) + | L-Arg (300 mg/kg) |
| | HBOC-201 (3.4 | |
| | ml/kg) + L-Arg | |
| 7 | 20% PEG-20k (3.4 ml/kg) + | D-Arg (300 mg/kg) |
| | HBOC-201 (3.4 | |
| | ml/kg) + D-Arg | |
| 8 | 20% PEG-20k (3.4 ml/kg) + | L-Arg + L-NAME |
| | HBOC-201 (3.4 | |
| | ml/kg) + L-Arg + L-NAME | |

L-Arg (300 mg/kg, IV) is a precursor substrate for nitric oxide synthase (NOS), D-Arg (300 mg/kg, IV) is the biochemically inactive racemate of L-Arg, and therefore, a competitive inhibitor of NOS, L-NAME (L-Nitro Arginine Methyl Ester, 10 mg/kg, IV) is a competitive antagonist of all forms of NOS. HBOC solution is administered immediately after PEG-20 k to represent the “worst case” NO scavenging scenario because the tissues still have significant oxygen debt. NO related drugs (L-Arg, D-Arg, and L-NAME) are all given I.V. in a 300 μl volumes of LR solution. N=10 rats per group.

In both Part 1 and Part 2, the primary measured outcomes include the rate of oxygen debt repayment on the first operative day and the re-accumulation of new oxygen debt the following days in rats that survive the initial 5-hour intra-operative observation period. We have demonstrated that shocked rats resuscitated with just PEG-20 k IV solution survive, where other groups including those resuscitated with whole blood do not but they begin to re-accumulate lactate a day after they recover. As described in Example 2, we have modified our previous shock model to include re-bleeding the animal 30 minutes after PEG-20 k resuscitation by removing 10% of the pre-shock estimated blood volume through a venous jugular catheter, centrifuging it and then re-administering the plasma intravenously. The model was designed to further reduce the oxygen carrying capacity of the resuscitated rat and also imitate the clinical scenario of uncontrolled rebleeding after resuscitation. In these rats, lactate measured from the tail at 12 hr after resuscitation returned to normal baseline levels but oxygen debt started to re-accumulate with lactate going up over the next 24 hours which resulted in the animals' death/euthanasia 24-48 hours later. Preventing this O₂ debt re-accumulation is a major primary outcome. Oxygen debt is estimated by plasma lactate concentrations. Other secondary outcomes measured in each experiment include MAP, blood labs, intestinal microcirculation measured with orthogonal polarization spectral imaging (OPSI) through a small enterotomy in the distal ileum, plasma Th1 cytokines, terminal bowel histology analysis, and subject survival time. Finally, local tissue production of nitric oxide, nitrosothiols, and nitric oxide metabolites is assessed from serial bowel mucosa biopsies using incubations of tissue in a Dubanoff metabolic incubator and a Sievers-Style chemiluminescence NO analyzer.

Nitric oxide metabolism is critical in early resuscitation from severe shock. Ischemia during shock sensitizes organs and tissue to local reductions in NO bioavailability. Therefore, the detection of better resuscitation outcomes with a lag time between the end of PEG-20 k resuscitation and the beginning of the HBOC infusion, indicates that the lag is beneficial secondary to time for debt repayment by PEG-20 k administered first. This is further supported by debt repayment at these later times as measured by plasma lactate concentration. The farther past PEG infusion, the lower the lactate falls. We identify a critical point of metabolic repair where reductions of NO levels due to the presence of free hemoglobin from the HBOC no longer negatively affect tissue reperfusion. This is the “wait it out” approach and is based in previous evidence.

In addition, to replenish NO synthesis capacity following depletion of endothelial stores of NO from HBOC administration, NO synthesis during LVR is addressed by administering the nitric oxide synthetase substrate L-Arginine in large amounts to drive increased endothelial NO production before HBOCs are given to compensate for the oxidative loss of local NO by the HBOC. Crystalloid resuscitation after severe hemorrhagic shock is dramatically improved with L-Arginine, and this effect is due to preservation of bowel perfusion and barrier function. The effects are due to NO synthesis since they are blocked by the competitive nitric oxide synthase (NOS) inhibitors D-Arginine and L-NAME. Improvements are seen by adding L-Arginine before HBOC and this effect is blocked by D-Arginine or L-NAME. Thus, L-Arginine is useful in supporting NO synthesis during resuscitation with HBOC-201.

In addition, or optionally, a second NO “sink” is provided by administration of nitrosothiolated fibrinogen or albumin. Local NO measurements (e.g. using a chemiluminescence analyzer and in-vitro tissue culture systems) validate the local NO availability under these various mitigation conditions.

In summary, the results show that delaying the time to administer the HBOC to increase oxygen carrying capacity mitigates the need for a nitric oxide replacement strategy since the PEG-20 k provides the initial oxygen debt repayment early by increasing capillary flow independent of exogenous hemoglobin. The data also shows that a rapid sequential administration of PEG-20 k solution first followed by an HBOC containing L-Arginine works too by providing simultaneous hemoglobin with exogenous nitric oxide through substrate level stimulation of nitric oxide by endothelial nitric oxide synthetase metabolism.

### Example 4. Restoration of Coagulation and Platelet Function Capacity Following PEG-20 k Resuscitation From Severe Hemorrhagic Shock

The hypocoagulable state and functional hypofibrinogenemia that exists immediately after severe blood loss and PEG-20 k resuscitation is mitigated by the concurrent addition of reconstituted lyophilized human fibrinogen (RiaSTAP®). The effects of severe blood loss after hemorrhage leads to direct depletion of clotting factors including fibrinogen. Furthermore, resuscitation with PEG-20 k IV solution causes rapid and long acting volume expansion by the osmotic movement of isotonic fluid from the intracellular and interstitial spaces back into the intravascular space, which further dilutes fibrinogen concentrations. In addition, a minor coagulopathy on TEG using PEG-20 k temporarily inhibits platelet cross-linking in the newly formed clot. Our best evidence is that this is caused by a nonspecific passivation of the platelet IIb/IIIa receptor to competitively inhibit binding to fibrinogen. Together, these forces tend to dilute out plasma fibrinogen concentrations that likely contribute to both the transient hypocoagulable state and the functional hypofibrinogenemia seen after PEG-20 k resuscitation in severe blood loss. Therefore, it makes sense that a simple add back of fibrinogen to the system after PEG-20 k IV solution resuscitation will correct the defect and improve coagulation and platelet function. Since human fibrinogen (e.g., RiaSTAP®) exists as a stable concentrate, it becomes part of the strategy for resuscitation in far forward field units with long projected evacuation times to comprehensively treat severe hemorrhagic shock by rapidly restoring tissue perfusion (PEG-20 k) while maintaining proper coagulation and platelet function with stable fibrinogen reconstitution.

### Part 1.

Changes in coagulation and platelet function is measured in the controlled lethal shock model in adult anesthetized rats in groups that receive PEG-20 k IV solution alone and those that receive PEG-20 k IV solution and supplemental human fibrinogen (200 mg/kg, IV, in 300 μl LR). The control group also receives 300 ul LR without RiaSTAP®. Samples of whole rat blood (1.0-ml) are taken at baseline and at various times after resuscitation for thromboelastography (TEG) analysis of coagulation and platelet function. Other assays are done to measure fibrinogen levels, intrinsic and extrinsic coagulation pathway coagulation, and platelet mapping studies are conducted to answer some mechanistic questions about platelet function defects. Samples are collected out to 4 hours, which is the time when coagulation and platelet function begins to return to baseline levels after PEG-20 k resuscitation. The groups used are listed in Table 3. Each group uses 10 adult Sprague-Dawley rats.

TABLE 3

| Group | LVR solution |
|---|---|
| 1 | Lactated Ringer's solution (6.8 ml/kg) |
| 2 | 20% PEG-20k (3.4 ml/kg) + 3.4 ml/kg LR |
| | vehicle |
| 3 | 20% PEG-20k (3.4 ml/kg) + 70 mg/kg RiaSTAP ® |
| | (in 3.4 ml/kg LR) |
| 4 | 20% PEG-20k (3.4 ml/kg) + 70 mg/kg albumin |
| | (in 3.4 ml/kg LR) |

### Part 2. Fibrinogen in Uncontrolled Hemorrhage

Coagulation and platelet function outcomes are tested by using a clinically relevant model of mixed polytrauma hemorrhagic shock that involves severe bleeding from both controllable and uncontrollable sources. Our lab has developed a rodent model of severe hemorrhagic shock where tail bleeding (controlled source) is combined with blood loss from an abdominal penetrating injury to the spleen (splenic laceration). This model fully tests the efficacy of fibrinogen reconstitution after PEG-20 k IV solution resuscitation. The groups of rats used in the uncontrolled hemorrhagic shock mode are shown in Table 3.

The main outcomes for part 1 in the controlled hemorrhage model are coagulation and platelet function measurement via thromboelastography (TEG) and ROTEM, assays for Fibrinogen concentration and function, PTINR, APTT INR, Thrombin release assays (CAT), platelet numbers, optical platelet aggregometry, platelet IIb/IIIa and CD62 expression using flow cytometry, and platelet mapping studies. The main outcomes for part 2 in the uncontrolled hemorrhage model include blood loss, survival time, physiological, coagulation, and metabolic outcomes.

These studies test the ability of fibrinogen add back to correct many of the coagulopathies induced by both severe hemorrhage and by PEG-20 k IV solution. The coagulopathies from PEG-20 k are mainly dilutional coagulopathy and a direct effect of PEG-20 k binding to platelet fibrinogen receptors to attenuate platelet crosslinking and clot strength. Fibrinogen add back left shifts the competitive effects of PEG-polymer binding to fibrinogen receptors. More importantly, the added fibrinogen serves to increase coagulation times by simple replacement of lost fibrinogen from the hemorrhage, which is seen by positive changes in INR, thrombin release, and platelet aggregation on TEG (MA). In uncontrolled hemorrhage models, fibrinogen reduces bleeding as evidenced by lower bleeding times, lower blood loss volumes, and improved physiological outcomes such as blood pressure, plasma lactate, heart rate, and survival.

In addition, or optionally, other factors and enzymes in the intrinsic and extrinsic coagulation pathways also require replacement. Mixtures of fibrinogen concentrate with freeze dried plasma preparations are used to gain additional coagulation function.

In summary, the results show that in controlled or uncontrolled hemorrhagic shock models, the addition of concentrated fibrinogen after resuscitation with PEG-20 k IV solution significantly improves coagulation outcomes and decreases uncontrolled bleeding times and volumes.

### Example 5. The Clinical Effect of Restoring Both Oxygen Carrying Capacity (HBOC+L-Arg) and Fibrinogen Concentrations to PEG-20 k Resuscitated Swine Using a Survival Mixed Controlled and Uncontrolled Blood Loss Model

Reconstitution of oxygen carrying capacity and coagulation and platelet function capacity with field stable additives significantly improves resuscitation outcomes in a relevant pre-clinical model of battlefield shock-trauma and prolonged field care. The gold standard for trauma resuscitation is rapid administration of fresh whole autologous blood or a combination of such blood products that restores a) lost intravascular volume, b) lost oxygen carrying capacity (hemoglobin laden red blood cells), and c) lost or consumed coagulation proteins and factors. But fresh whole autologous blood is still ineffective if the capillary microcirculation is swollen shut as a consequence of the initial tissue ischemia that occurs as a consequence of initial blood loss or from cardiovascular adjustments secondary thereto.

The “PEG first” concept was developed where PEG-20 k IV solution is first administered to shocked patients to open up the capillary beds in vital organs and tissues that swell shut after shock followed by administration of fresh whole autologous blood or blood products. The polymer opens the capillaries and the fresh blood is then able to deliver oxygen carrying capacity to the ischemic cells. This creates a new gold standard because the problem of no reflow is fixed by administration of the PEG-20 k IV solution.

However, in austere battlefield conditions, even this approach is not always available because it still relies on a blood donor in the field. The donors may not be readily available or it may be undesirable to send combat ready special forces on missions after blood donation, which necessarily compromises their physical and mental acuity and robustness in the field. Therefore, we have reconstituted a stable product and developed a protocol for its use that leverages the “PEG First” doctrine of field resuscitation that is effective, usable in the field, and independent of blood donors. This will safely prolong field and evacuation times to where the golden hour now becomes the golden day.

The general concept is to improve the outcomes of PEG-20 k IV solution resuscitation for traumatic hemorrhagic shock by functionally reconstituting the solution with field stable additives that add oxygen carrying capacity (hemoglobin) and coagulation factors. The Examples above teach us the details and best practices to safely do this. Therefore, the new protocol using PEG-first followed by HBOC under nitric oxide sparing conditions, and then fibrinogen is tested in a porcine survival model of traumatic hemorrhagic shock involving both controlled and uncontrolled bleeding. The groups of 30-35 kg swine are shown in Table 5.

| Group | LVR Solution |
|---|---|
| 1 | Whole fresh autologous blood (6.8 ml/kg) + 200 ml |
| | LR++ |
| 2 | 10% PEG-20k (6.8 ml/kg) + 200 ml LR |
| 3 | 20% PEG-20k (3.4 ml/kg) + L Arg (300 mg/kg) in |
| | 200 ml LR + HBOC-201 |
| | (¾ ml/kg) |
| 4 | 20% PEG-20k (3.4 ml/kg) + 200 mg/kg RiaStap ® in |
| | 3.4 ml/kg LR + 200 ml |
| | LR |
| 5 | 20% PEG-20k (3.4 ml/kg) + 200 mg/kg RiaStap ® in |
| | 150 ml LR + L Arg (300 |
| | mg/kg) in 50 ml LR + HBOC-201 (¾ ml/kg) |

++150 ml LR is the minimum volume needed to solubilize the RiaStap (100 ml) and the L-Arginine (50 ml). Therefore, an additional 150 ml LR is infused in all of the other groups too.

Each group consists of 10 pigs. Swine are anesthetized and a midline laparotomy is performed to remove the spleen. A 4 cm hemisection of the left lateral lower lobe of the liver is made with scissors to induce uncontrolled bleeding. Then, arterial blood is removed (up to 37 ml/kg) and hypotension is maintained until the plasma lactate reaches 7-8 mM, after which the LVR solutions are administered. A loop of distal ileum is exposed and a 1 cm enterotomy is made along the anti-mesenteric border to access the mucosal surface for hourly recordings of capillary blood flow by OPSI and for biopsies of bowel mucosa at various times before shock and after resuscitation. The biopsies are used for histological analysis and for nitric oxide metabolism studies ex-vivo in a tissue culture system. After resuscitation, pigs are closed and followed acutely for 4 hours. Those that survive are recovered from anesthesia and euthanized 7 days later after collection of terminal physiological, metabolic, coagulation, platelet function, and histological data.

Swine response to shock and resuscitation over the first 4 hours includes central hemodynamics including mean arterial pressure, heart rate, cardiac output (transthoracic cardiac ultrasound imaging), microcirculation in the terminal ileum (Orthogonal Polarization Spectral Imaging, OPSI), plasma lactate and other labs, oxygen delivery (Fick), and survival time. For survivors, outcomes include neurological function, survival and physiological and organ histological data after 7-days (post-mortem). Coagulation and platelet function is measured by TEG at baseline, every hour after resuscitation, up to 4 hours, 24 hours after survival, and before euthanasia after 7-days (for those that survive).

Use of this well-characterized swine model determines how both optimizations to PEG-20 k IV solution work in human prehospital resuscitation. The analysis is direct and straightforward because a treatment effect is first generated by comparing the benefits of PEG-20 k solution with fresh autologous whole blood.

Optimized HBOC-201 with fibrinogen concentrate is used to serially reconstitute oxygen carrying capacity (HBOC) and coagulation function, respectively, to assess the benefits of both in the pre-clinical model. The assessments of the groups is based on survival, cardiovascular performance outcomes, bleeding volumes, and metabolism (oxygen debt repayment). The uncontrolled hemorrhage component from the liver laceration provides the coagulation components missing from previous swine models.

The contributions of the individual components (fibrinogen and HBOC) to the solution are determined in subtraction experiments where fibrinogen and HBOC are sequentially removed to determine the effect of each on outcomes.

Cross over actions and potentiation occur. For example, the addition of extra oxygen carrying capacity (HBOC) to enhance coagulation, even without fibrinogen add back, prevents downstream coagulopathies due to local ischemia.

In some aspects, fibrinogen alone enhances tissue oxygen debt repayment by preventing re-bleeding after resuscitation.

In addition, or optionally, nitrosothiolation of the HBOC before use or nitrosothiolation of the fibrinogen is/are administered to enhance local NO sinks, as are fibrinogen mixed with freeze-dried plasma preparations that are also stable in the field.

In summary, the results show that in a severe shock model, superior survival, oxygen debt repayment and maintenance, and improved hemostatic outcomes are realized when these components are used together in the proper order, compared to any single component used alone or compared to current standard of care.

### Example 6

Capillary perfusion was measured by Orthogonal Polarization Spectral Imaging (OPSI) in the distal ileal mucosa in shocked swine. The results are presented in FIG. 5. Values of capillary perfusion (PPV) were obtained before shock, after shock, and at various times after resuscitation with equal volumes (6.8 ml/kg) of 10% PEG-20 k IV Solution, Lactated Ringers vehicle, and fresh autologous whole blood. Values shown are mean+/−Standard error of the mean from 5-6 independent swine shock studies per group. These data clearly show the direct comparisons of PEG-20 k with whole blood where PEG-20 k solution with low oxygen carrying capacity (0.41 ml O2/dl) has a much higher capillary perfusion compared to whole blood that carries much more oxygen (21 ml O2/dl). Therefore, these data demonstrate the value of administration of PEG-20 k IV solution first during shock resuscitation followed by a hemoglobin-based oxygen carrier (HBOC) to maximize the positive attributes of both solutions when given in the proper order. PEG-20 k solution first opens capillaries to accommodate subsequently administered oxygen carriers, which maximizes oxygen transfer to ischemic tissues after shock. HBOCs like HBOC-201 are preferred oxygen carriers compared to whole blood because they are stable solutions that can be shelf stored without refrigeration for prehospital use in the field or for use in community hospital mass casualty situations where whole blood is not available in sufficient quantities.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Bellamy RF. The causes of death in conventional land warfare: implications for combat casualty care research. MilMed. 1984; 149(2):55-62.
2. Champion HR, Bellamy RF, Roberts CP, Leppaniemi A. A profile of combat injury. JTrauma. 2003; 54(5 Suppl):S13-S9.
3. Holcomb JB. Fluid resuscitation in modern combat casualty care: lessons learned from Somalia. JTrauma. 2003; 54(5 Suppl):S46-S51.
4. National Center for Injury Prevention and Control. Web-based Injury Statistics Query and Reporting System (WISQARS) 2013 [updated 2013. Available from: the website located at www.cdc.gov/injury/wisqars/.
5. Wang A, Xiang H. U.S. Leading Causes of Death and Years of Potential Life Lost, 1981-2019: Implications for Surgical Research. J Surg Res. 2022.
6. Kauvar DS, Lefering R, Wade CE. Impact of hemorrhage on trauma outcome: an overview of epidemiology, clinical presentations, and therapeutic considerations. JTrauma. 2006; 60(6 Suppl):S3-11.
7. Heckbert SR, Vedder NB, Hoffman W, Winn RK, Hudson LD, Jurkovich GJ, Copass MK, Harlan JM, Rice CL, Maier RV. Outcome after hemorrhagic shock in trauma patients. JTrauma. 1998; 45(3):545-9.
8. Franklin GA, Boaz PW, Spain DA, Lukan JK, Carrillo EH, Richardson JD. Prehospital hypotension as a valid indicator of trauma team activation. JTrauma. 2000; 48(6):1034-7.
9. Riha GM, Kunio NR, Van PY, Hamilton GJ, Anderson R, Differding JA, Schreiber MA. Hextend and 7.5% hypertonic saline with Dextran are equivalent to Lactated Ringer's in a swine model of initial resuscitation of uncontrolled hemorrhagic shock. JTrauma. 2011; 71(6):1755-60.
10. Riha GM, Kunio NR, Van PY, Kremenevskiy I, Anderson R, Hamilton GJ, Differding JA, Schreiber MA. Uncontrolled hemorrhagic shock results in a hypercoagulable state modulated by initial fluid resuscitation regimens. JTrauma AcuteCare Surg. 2013; 75(1):129-34.
11. Montroy J, Lavallee LT, Zarychanski R, Fergusson D, Houston B, Cagiannos I, Morash C, Tinmouth A, Hutton B, Mallick R, Flaman A, Breau RH. The Top 20 Surgical Procedures Associated with the Highest Risk for Blood Transfusion. Br J Surg. 2020; 107(13):e642-e3.
12. Miskeen E, Omer Yahia AI, Eljack TB, Karar HK. The Impact of COVID-19 Pandemic on Blood Transfusion Services: A Perspective from Health Professionals and Donors. J Multidiscip Healthc. 2021; 14:3063-71.
13. Pusateri AE, Malloy WW, Sauer D, Benov A, Corley JB, Rambharose S, Wallis L, Tiller MM, Cardin S, Glassberg E, Weiskopf RB. Use of Dried Plasma in Prehospital and Austere Environments. Anesthesiology. 2022; 136(2):327-35.
14. Mok G, Hoang R, Khan MW, Pannell D, Peng H, Tien H, Nathens A, Callum J, Karkouti K, Beckett A, da Luz LT. Freeze-dried plasma for major trauma—Systematic review and meta-analysis. J Trauma Acute Care Surg. 2021; 90(3):589-602.
15. Khoraki J, Wickramaratne N, Kang HS, Xu H, Archambault C, Blocher C, Li R, Liebrecht L, Aboutanos M, Mangino MJ. Superior Survival Outcomes of a Polyethylene Glycol-20 k Based Resuscitation Solution in a Preclinical Porcine Model of Lethal Hemorrhagic Shock. Ann Surg. 2020.
16. Parrish D, Lindell S, Reichstetter H, Aboutanos M, Mangino MJ. Cell impermeant based low volume resuscitation in hemorrhagic shock: A biological basis for injury involving cell swelling. Ann Surg. 2014.
17. Plant V, Parrish DW, Limkemann A, Ferrada P, Aboutanos M, Mangino MJ. Low-Volume Resuscitation for Hemorrhagic Shock: Understanding the Mechanism of PEG-20 k. J Pharmacol Exp Ther. 2017; 361(2):334-40.
18. Plant V, Limkemann A, Liebrecht L, Blocher C, Ferrada P, Aboutanos M, Mangino MJ. Low-volume resuscitation using polyethylene glycol-20 k in a preclinical porcine model of hemorrhagic shock. J Trauma Acute Care Surg. 2016; 81(6):1056-62.
19. Parrish D, Plant V, Lindell SL, Limkemann A, Reichstetter H, Aboutanos M, Mangino MJ. New low-volume resuscitation solutions containing PEG-20 k. J Trauma Acute Care Surg. 2015; 79(1):22-9.
20. Arora TK, Malhotra AK, Ivatury R, Mangino MJ. L-arginine infusion during resuscitation for hemorrhagic shock: impact and mechanism. JTrauma AcuteCare Surg. 2012; 72(2):397-402.
21. Mees N, Southard JH, Belzer FO. Inhibition of ischemic induced cellular swelling in kidney cortex tissue by lactobionate anions. JTrauma. 1982; 22(2):118-20.
22. Hishikawa K, Nakaki T, Suzuki H, Saruta T, Kato R. L-arginine-induced hypotension. Lancet. 1991; 337(8742):683-4.

1. An intravenous (IV) delivery device comprising
a first container containing a first solution comprising 18,000-100,000 Da PEG polymers and, optionally, 1,000 up to 18,000 Da PEG polymers in a first physiologically compatible liquid carrier; and
a second container containing a second solution comprising a hemoglobin-based oxygen carrier (HBOC) in a second physiologically compatible liquid carrier;
wherein the first and second containers are each configured to receive an IV drip.

2. The IV delivery device of claim 1, wherein the 18,000-100,000 Da PEG polymers are 20,000 Da PEG polymers (PEG-20 k) and the 1,000 up to 18,000 Da PEG polymers are 8,000 Da PEG polymers (PEG 8 k).

3. The IV delivery device of claim 1, wherein the second solution further comprises an agent that mitigates NO oxidation.

4. The IV delivery device of claim 3, wherein the agent that mitigates NO oxidation is L-arginine.

5. The IV delivery device of claim 1, wherein the first and second containers are flexible IV bags.

6. The IV delivery device of claim 1, wherein the first and second containers are attached via a liquid impermeable seam.

7. The IV delivery device of claim 1, further comprising a container comprising physiological saline solution.

8. The IV delivery device of claim 1, wherein the first and second physiologically compatible liquid carriers are the same or different.

9. The IV delivery device of claim 1, wherein the first and second containers each contain from 25-500 mls of liquid, and wherein and amount of the liquid contained in the first and second containers are the same or different.

10. The IV delivery device of claim 1, wherein:
the first solution comprises 250 ml of 200 mg/ml (20%) PEG-20 k and, optionally, 20 mg/ml (2%) PEG-8 k in the first physiologically compatible liquid carrier; and/or
the second solution comprises 250 ml of 12 mg/ml HBOC and, optionally, 88.2 mg/ml L-arginine in the second physiologically compatible liquid carrier.

11. The IV delivery device of claim 1, wherein the first and second physiologically compatible liquid carriers comprise one or more of NaCl, sodium lactate, KCl and CaCl₂.

12. The IV delivery device of claim 1, wherein the first and second physiologically compatible liquid carriers comprise 1.2 mg/ml NaCl, 0.62 mg/ml sodium lactate, 0.06 mg/ml KCl and 0.04 mg/ml of CaCl₂.

13. The IV delivery device of claim 1, further comprising a third container containing a third solution comprising at least one hemostasis agent in a third physiologically compatible liquid carrier.

14. The IV delivery device of claim 13, wherein the at least one hemostasis agent is lyophilized fibrinogen concentrate or lyophilized plasma.

15. The IV delivery device of claim 13, wherein:
the third solution comprises,
100-250 ml of reconstituted fibrinogen (10-50 mg/ml) in the third physiologically compatible liquid carrier, and/or
100-250 ml of reconstituted lyophilized plasma in the third physiologically compatible liquid carrier.

16. A method of treating a subject suffering from blood loss, comprising
i) administering to the subject a therapeutically effective amount of a first solution comprising 18,000-100,000 Da PEG polymers and, optionally, 1,000 up to 18,000 Da PEG polymers in a first physiologically compatible liquid carrier, and then
ii) administering to the subject a therapeutically effective amount of a second solution comprising a hemoglobin-based oxygen carrier (HBOC).

17. The method of claim 16, wherein the18,000-100,000 Da PEG polymers are 20,000 Da PEG polymers (PEG-20 k) and the 1,000 up to 18,000 PEG Da polymers are 8,000 Da PEG polymers (PEG 8 k).

18. The method of claim 16, wherein the second solution further comprises an agent that mitigates NO oxidation.

19. The method of claim 18, wherein the agent that mitigates NO oxidation is L-arginine.

20. The method of claim 16, wherein
the first solution comprises 250 ml of 200 mg/ml PEG-20 k and, optionally, 20 mg/ml PEG-8 k in the first physiologically compatible liquid carrier; and
the second solution comprises 250 ml of 12 mg/ml HBOC and, optionally, 84 mg/ml L-arginine in the second physiologically compatible liquid carrier.

21. The method of claim 16, further comprising
iii) administering to the subject a therapeutically effective amount of a third solution comprising at least one hemostasis agent, wherein step iii) of administering is performed after step ii) of administering.

22. The method of claim 21, wherein the at least one hemostasis agent is fibrinogen or reconstituted lyophilized plasma.

23. The method of claim 21, wherein the third solution comprises
100-250 ml of 10-50 mg/ml fibrinogen in the third physiologically compatible liquid carrier, and/or
100-250 ml of reconstituted lyophilized plasma in the third physiologically compatible liquid carrier.

24. The method of claim 21, wherein the third solution is administered after the first solution and before the second solution.

25. A kit comprising,
the IV device of claim 1, and
an IV infusion set.
